(19) United States
(12) Patent Application Publication
(10) Pub. No.: US 2018/0092509 A1
(43) Pub. Date: Apr. 5, 2018

(54) IMAGE RECORDING DEVICE

(71) Applicant: OLYMPUS CORPORATION, Tokyo (JP)

(72) Inventor: Masahide YAMAKI, Hachioji-shi (JP)

(73) Assignee: OLYMPUS CORPORATION, Tokyo (JP)

(21) Appl. No.: 15/822,000

(22) Filed: Nov. 24, 2017

Related U.S. Application Data
(63) Continuation of application No. PCT/JP2016/062936, filed on Apr. 25, 2016.

(30) Foreign Application Priority Data
May 27, 2015 (JP) 2015-107807

(51) Int. Cl. A61B 1/00; A61B 18/14; A61B 1/04; A61M 13/00; A61B 1/06; H04N 5/77; G11B 27/02; G11B 27/19; G11B 27/34

(52) U.S. Cl. CPC A61B 1/0002; A61B 18/14; A61B 1/04; A61M 13/003; A61B 1/0661; G16H 30/20; G11B 27/02; G11B 27/19; G11B 27/34; A61B 1/00009; A61B 2018/1412; H04N 5/77

(57) ABSTRACT

An image recording device includes a first movie generation section configured to, based on the medical image, generate a first movie of a first image quality for a first use, and output a movie of a second image quality higher than the first image quality, a detection section configured to detect at least one of a state signal from an external appliance and a detection timing based on an operation signal from an operation section, a duplication section configured to generate a duplicate of the movie of the second image quality at the detection timing, a second movie generation section configured to generate a second movie for a second use based on the duplicated movie, and a recording section configured to record the first movie, and to record the second movie in association with the reference time information.

### CROSS REFERENCE TO RELATED APPLICATION

This application is a continuation application of PCT/JP2016/062936 filed on Apr. 25, 2016 and claims benefit of Japanese Application No. 2015-107807 filed in Japan on May 27, 2015, the entire contents of which are incorporated herein by this reference.

### BACKGROUND OF THE INVENTION

### 1. Field of the Invention

The present invention relates to an image recording device configured to record an image obtained by a medical device such as an endoscope.

### 2. Description of the Related Art

Conventionally, endoscopes are widely used in a medical field and the like. In recent years, due to increased image quality (realization of high-vision) of endoscopes, tissues in an abdominal cavity, such as abdominal structures and vessel courses, can be clearly seen, and endoscopic surgeries can be performed more safely and reliably.

In a medical institution, recording of various medical images, such as endoscopic images, ultrasound images, and X-ray images, is performed by combining many modalities such as an endoscope, X-rays, and an ultrasound analysis apparatus. A conventional image recording device configured to record medical images is capable of outputting recorded data in various formats according to use. For example, during a surgery, recording can be performed in a format allowing computer editing in real time by using a semiconductor recording device, such as a USB memory, or an optical medium. Furthermore, recording can be performed in a format allowing reproduction by a general-purpose video player by using an optical medium. Moreover, a medical image may be transferred to a server via a network and be recorded so as to allow sharing of data.

Now, it is conceivable for cases to be recorded for the purpose of allowing medical images to be used as backups of evidence images or the like, or as educational materials. For example, a recorded image of an important incision scene in a case can be shared in an academic or in-hospital conference so as to train young doctors. Moreover, an endoscopic procedure or the like may be recorded to be used for the Endoscopic Surgical Skill Qualification System, and qualification for the procedure may be decided based on the recorded image.

In the case of recording for backup, a long case has to be entirely recorded, and thus, recording is sometimes performed of a relatively low image quality taking a recording capacity into account. Furthermore, in the case where a case is recorded as educational material, sometimes only a necessary part is recorded of a highest image quality so as to facilitate observation of the case based on the recorded image.

An image recording device which is configured to record medical images is not necessarily placed at a position, in a surgery room or the like, where operation can be easily performed or setting of recording can be easily checked. Even if recording setting can be performed by a remote control device or the like, the image recording device is generally placed at a position where an image being recorded cannot be easily checked. Accordingly, although a recording start operation and a stop operation can be performed during a surgery for each of necessary scenes desired to be recorded for educational purposes or an academic conference presentation, a method of entirely recording one case is often adopted taking into account a great risk of forgetting to resume recording.

Japanese Patent Application Laid-Open Publication No. 2011-36372 discloses a device which is configured to detect a current flowing through an electric scalpel during use of the electric scalpel and to detect a change in biological information to perceive features to thereby create a digest operation image collecting only the specific features.

### SUMMARY OF THE INVENTION

An image recording device according to an aspect of the present invention includes a medical image capturing section configured to capture a medical image from a controlled appliance, a single encoder configured to encode the medical image being inputted into a video signal in a predetermined image format, a first movie generation section configured to, based on the medical image encoded by the encoder and associated with reference time information, generate a first movie of a first image quality for a first use, and output a movie of a second image quality higher than the first image quality, the movie of the second image quality being the inputted medical image; a detection section configured to detect at least one of a first detection timing based on a state signal from an external appliance and a second detection timing based on an operation signal from an operation section; a variable movie buffer memory configured to accumulate the movie of the second image quality outputted from the first movie generation section, over a predetermined period of time; a duplication section configured to generate a duplicate of the movie of the second image quality accumulated in the variable movie buffer memory, at a detection timing detected by the detection section; a second movie generation section configured to generate a second movie for a second use different from the first movie, based on a movie of the second image quality duplicated by the duplication section; and a recording section configured to record the first movie, and to record the second movie in association with the reference time information.

### BRIEF DESCRIPTION OF THE DRAWINGS

FIG. 1 is a block diagram showing an image recording device according to a first embodiment of the present invention;

FIG. 2 is an explanatory diagram showing a state of a surgery room where the image recording device in FIG. 1 is installed;

FIG. 3 is an explanatory diagram for describing an example of a table of an amount of time shift;

FIG. 4 is a flowchart for describing an operation according to the first embodiment;

FIG. 5 is an explanatory diagram for describing a second embodiment of the present invention;

FIG. 6 is a flowchart for describing an operation according to the second embodiment;

FIG. 7 is an explanatory diagram for describing the operation according to the second embodiment;

FIG. 8 is a block diagram showing a modification;

FIG. 9 is a flowchart for describing an operation according to the modification;

FIG. 10 is a block diagram for describing a third embodiment of the present invention; and

FIG. 11 is a block diagram for describing a fourth embodiment of the present invention.

### DETAILED DESCRIPTION OF THE PREFERRED EMBODIMENT(S)

Hereinafter, embodiments of the present invention will be described in detail with reference to the drawings.

### First Embodiment

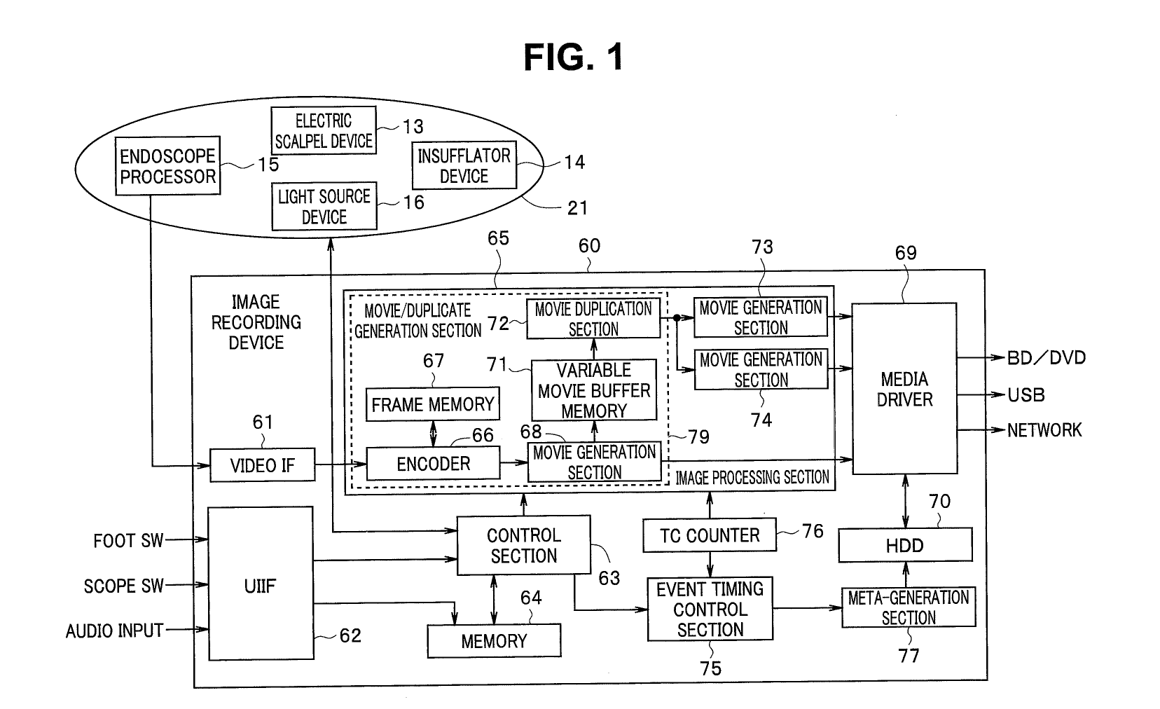

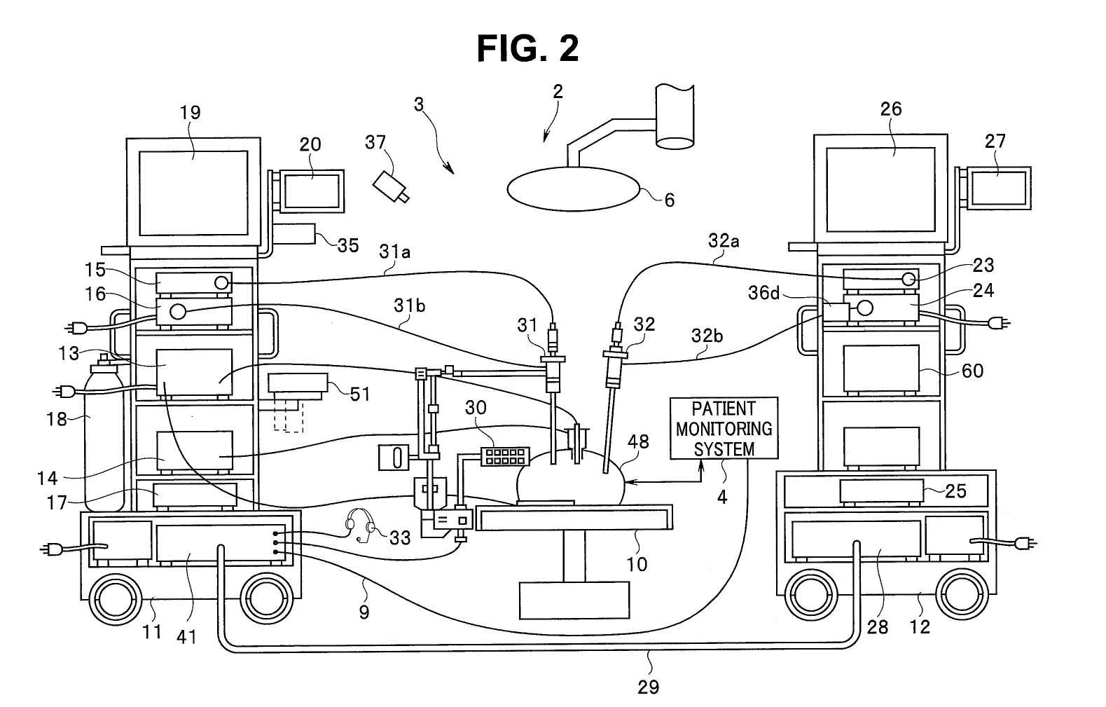

FIG. 1 is a block diagram showing an image recording device according to a first embodiment of the present invention. Moreover, FIG. 2 is an explanatory diagram showing a state of a surgery room where the image recording device in FIG. 1 is installed.

First, installation of an image recording device 60 in a surgery room 2 will be described with reference to FIG. 2. As shown in FIG. 2, a medical system 3 arranged in the surgery room 2 includes a system controller 41 configured to control medical appliances such as an operating table 10 where a patient 48 is to lie down and an electric scalpel device 13. A first cart 11 and a second cart 12 are provided in the surgery room 2, and the system controller 41 is placed on the first cart 11.

Moreover, a gas cylinder 18 filled with carbon dioxide and medical appliances which are controlled devices are placed on the first cart 11, the medical appliances including the electric scalpel device 13, an insufflator device 14, an endoscope processor 15, a light source device 16, and a video recorder 17, for example. The endoscope processor 15 is connected to a first endoscope 31 via a camera cable 31a.

The light source device 16 is connected to the first endoscope 31 via a light guide cable 3 lb. Moreover, a display device 19, a first central display panel 20, an operation panel 51 and the like are placed on the first cart 11. For example, the display device 19 is a TV monitor configured to display an endoscopic image and the like.

The central display panel 20 is a display means capable of selectively displaying any data during a surgery. The operation panel 51 is configured of a display screen, such as a liquid crystal display, and a touch sensor provided on the display screen in an integrated manner, for example, and is a central operation device to be operated by a nurse or the like in a non-sterile area.

The operating table 10, a shadowless lamp 6, the electric scalpel device 13, the insufflator device 14, the endoscope processor 15, the light source device 16, and the video recorder 17 are connected to the system controller 41 as a central control device via communication lines (not shown). A headset microphone 33 can be connected to the system controller 41, and the system controller 41 is capable of recognizing audio inputted from the microphone 33, and of allowing control of each appliance by a voice of a surgeon.

Furthermore, an endoscopic image from the endoscope processor 15 is supplied to the image recording device 60. Moreover, the electric scalpel device 13, the insufflator device 14, the endoscope processor 15, the light source device 16, and the image recording device 60 are structured in a predetermined network 21 (see FIG. 1), and information indicating a state of each appliance is supplied to the image recording device 60. Note that communication lines of various communication standards, such as FlexRay, may be used for the network 21.

An RFID (radio frequency identification) terminal 35 capable of wirelessly reading/writing individual ID information of an object for an ID tag embedded in the first endoscope 31, a treatment instrument such as the electric scalpel device 13 or the like is further provided on the first cart 11.

A video processor 23, a light source device 24, an image processing device 25, a display device 26, and a second central display panel 27, which are controlled devices, are placed on the second cart 12. The video processor 23 is connected to a second endoscope 32 via a camera cable 32a. The light source device 24 is connected to the second endoscope 32 via a light guide cable 32b.

The display device 26 displays endoscopic images taken by the video processor 23, and the like. The second central display panel 27 is capable of selectively displaying any data during a surgery.

The video processor 23, the light source device 24, and the image processing device 25 are connected to a junction unit 28 placed on the second cart 12 by communication lines (not shown). Moreover, the junction unit 28 is connected to the system controller 41 placed on the first cart 11 by a junction cable 29.

The system controller 41 is thus allowed to control, in a central manner, the video processor 23, the light source device 24, and the image processing device 25 placed on the second cart 12, the electric scalpel device 13, the insufflator device 14, the endoscope processor 15, the light source device 16, and the video recorder 17 placed on the first cart 11, and the operating table 10. When communication is being performed between the system controller 41 and the devices, the system controller 41 is capable of displaying, on the display screen of the operation panel 51, a setting screen showing a setting state of a connected device, an operation switch and the like. Furthermore, the system controller 41 allows an operation input for changing a setting value or the like to be performed when a desired operation switch is touched and a predetermined region of a touch panel is operated.

A remote control 30 is a second central operation device to be operated by an operating surgeon or the like in a sterile area, and is capable of operating, via the system controller 41, other devices with which communication is established.

Furthermore, an infrared communication port (not shown), which is a communication means, is attached to the system controller 41. The infrared communication port is provided at a position near the display device 19 where infrared light can be easily radiated, for example, and the port and the system controller 41 are connected to each other by a cable.

The system controller 41 is connected to a patient monitoring system 4 by a cable 9, and the patient monitoring system 4 is capable of analyzing biological information, and of displaying an analysis result on a desired display device.

Note that a camera 37 configured to pick up images of a medical appliance, such as the operating table 10, and the like is further provided in the surgery room 2. By picking up an image of a medical appliance, such as the operating table 10, by the camera 37, and analyzing the picked-up image, an operation state may be determined. A determination result and a picked-up image of the camera 37 are supplied to the system controller 41.

FIG. 1 shows an example of a specific configuration of the image recording device 60 in FIG. 2.

The present embodiment describes an example where the endoscope processor 15 is used as a device for outputting a medical image. Note that as the device for outputting a medical image, the video processor 23 may be used, or both the endoscope processor 15 and the video processor 23 may be used. The endoscope processor 15 is capable of capturing an image from an endoscope (not shown), subjecting the image to image signal processing, and thereby generating a medical image such as an endoscopic image. The endoscope processor 15 is capable of outputting the endoscopic image as a high-definition image. The medical image from the endoscope processor 15 is supplied to a video IF 61 of the image recording device 60.

A UIIF 62 is also provided in the image recording device 60, and an operation signal generated by an operation by a surgeon is inputted to the UIIF 62. For example, operation signals based on operation of a foot switch (SW) by a surgeon, operation of a scope SW provided on an endoscope, audio input operation by a surgeon are inputted. The UIIF 62 receives an operation signal based on operation by a surgeon, and outputs the signal to a control section 63. Note that FIG. 1 shows an example where three types of operation signals are inputted, but various operation signals that are generated by an operation section which can be operated by a surgeon during a surgery may be used as operation signals to be inputted to the UIIF 62.

The control section 63 is capable of controlling each section of the image recording device 60. The control section 63 may be configured by a processor such as a CPU (not shown), and may control each section by operating according to a program stored in a memory 64.

The video IF 61 is an interface suitable for image transfer, and captures a medical image from the endoscope processor 15. Note that various terminals such as a DVI (digital visual interface) terminal, an SDI (serial digital interface) terminal, an RGB terminal, a Y/C terminal, and a VIDEO terminal may be adopted for the video IF 61. The video IF 61 is capable of capturing various medical images not only from the endoscope processor 15 but also from an ultrasound device, a surgical field camera, an X-ray observation device, an endoscope processor different from the endoscope processor 15, and the like.

A medical image captured by the video IF 61 is given to an encoder 66 in a movie/duplicate generation section 79 of an image processing section 65. The encoder 66 encodes an inputted medical image into a video signal in a predetermined image format by storing the inputted medical image in a frame memory 67 and reading the medical image at the same time and thereby performing encoding processing on the medical image. For example, the encoder 66 is capable of converting the inputted medical image into a video signal of MPEG2 format or MPEG-4AVC/H.264 format, or the like. The medical image from the encoder 66 is given to a movie generation section 68.

In the present embodiment, the movie generation section 68 is controlled by the control section 63 to generate a movie suitable for a first use set in advance, and to output the movie to a media driver 69. The media driver 69 as a recording section is capable of giving the movie generated by the movie generation section 68 to an external recording medium to have the movie recorded, and of giving the movie to a built-in hard disk drive (HDD) 70 to have the movie recorded. Note that FIG. 1 shows a BD (Blu-ray disc), a DVD, a USB, a server on a network and the like as external recording media, but other recording media may also be used.

For example, the control section 63 may set a record of evidence for a lawsuit as the first use to be set in the movie generation section 68, and in such a case, the movie generation section 68 is controlled such that a movie of a low image quality is generated, for example.

Conventionally, setting of an image quality and the like of a movie to be recorded is allowed, and moreover, start and end of recording of a movie may be controlled by using a foot switch output or an electric scalpel output, for example. However, to prevent forgetting to resume recording, a case often has to be entirely recorded of a high image quality, and in such a case, a task such as reducing the image quality for a record of evidence or editing for academic conference presentation has to be performed after recording. Moreover, in a case of controlling start and end of recording by using an electric scalpel output or the like, a surgery has to be entirely recorded as a record of evidence, by providing an encoder (recording device) of another system. In such a case, each of the encoders of two systems has to be operated, and a recording operation becomes burdensome.

Accordingly, in the present embodiment, at least one duplicate is generated from a medical image based on an output of the encoder 66, and a movie suitable for each use is generated from a generated duplicate. Moreover, in the present embodiment, start and end of recording of a movie based on a duplicate, and the use are controlled based on an operation signal from a surgeon or a state signal from an external appliance.

The movie generation section 68 is capable of giving an output of the encoder 66 to a variable movie buffer memory 71 without any change. A medical image is given to the variable movie buffer memory 71 from the movie generation section 68. Note that the medical image here is an image of a highest image quality which is outputted from the encoder 66. That is, the movie generation section 68 is capable of outputting not only a movie for the first use but also a movie of a maximum image quality from the encoder 66. The variable movie buffer memory 71 is a ring buffer having a capacity for accumulating medical images over a predetermined period of time, and configured to update and store sequentially inputted medical images. A movie duplication section 72 is controlled by the control section 63 to read a medical image from the variable movie buffer memory 71 to create a duplicate and to give the duplicate to movie generation sections 73, 74. The movie/duplicate generation section 79 is configured of the encoder 66, the frame memory 67, the movie generation section 68, the variable movie buffer memory 71, and the movie duplication section 72.

The movie generation section 73 is controlled by the control section 63 to generate a movie suitable for a second use and to output the movie to the media driver 69, and the movie generation section 74 is controlled by the control section 63 to generate a movie suitable for a third use and to output the movie to the media driver 69. The media driver 69 is capable of giving a movie generated by the movie generation sections 68, 73, 74 to an external recording medium and causing the external recording medium to have the movie recorded, and of giving the movie to the HDD 70 to have the movie recorded.

For example, the media driver 69 includes an optical drive device (not shown) configured to record a movie in and read a movie from an optical medium such as a Blu-ray disc, and a USB recording/reproduction section (not shown) configured to record a movie in and read a movie from a USB medium such as a USB memory. Moreover, the media driver 69 includes a network interface (not shown) configured to transfer a medical image to a not-shown network server to be recorded and reproduced, for example.

In the present embodiment, a timing of duplication by the movie duplication section 72 is controlled by the control section 63. The control section 63 as a detection section controls start and end of duplication by the movie duplication section 72 at a timing of an operation signal inputted to the UIIF 62 or a timing that is based on a state signal from an external appliance connected to the network 21. For example, the control section 63 may cause duplication by the movie duplication section 72 to start at a timing when the foot SW is pressed, and may cause duplication by the movie duplication section 72 to end at a timing when the foot SW is released, or may cause the movie duplication section 72 to start duplication or end duplication at each timing of pressing of the scope SW, for example.

Moreover, in the present embodiment, the control section 63 controls an address, in the variable movie buffer memory 71, where reading is to be performed by the movie duplication section 72, so as to enable time-shifted duplication where duplication of a medical image is retroactively performed. For example, even if a surgeon performs an operation to start duplication after occurrence of bleeding, a duplicate of a medical image of a bleeding region may be generated by the movie duplication section 72 starting reading of a medical image from a data portion picked up before bleeding and recorded in the variable movie buffer memory 71. Furthermore, the control section 63 may change an amount of time shift according to an operation by a surgeon. For example, by having a table describing the amount of time shift stored in the memory 64, the amount of time shift may be changed by a simple operation.

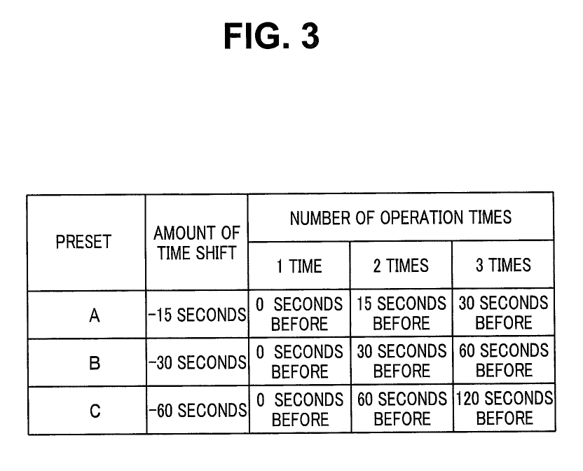

FIG. 3 is an explanatory diagram for describing an example of a table of the amount of time shift. FIG. 3 shows an example of a table including three types of presets with respect to the amount of time shift. A preset A indicates that the amount of time shift is changed by −15 seconds every time the foot SW is pressed. Likewise, presets B, C indicate that the amount of time shift is changed by −30 seconds and −60 seconds, respectively, every time the foot SW is pressed. In the example in FIG. 3, if the foot SW is pressed once, time shift is not performed, and a medical image from an image picked up at a timing of operation of the foot SW is duplicated.

For example, when the foot SW is pressed twice successively in a state where the preset A is set, the control section 63 performs control based on the table stored in the memory 64, such that duplication of a medical image is performed from an image picked up 15 seconds before the operation of the foot SW. Moreover, for example, when the foot SW is pressed three times successively in a state where the preset C is set, the control section 63 performs control based on the table stored in the memory 64, such that duplication of a medical image is performed from an image picked up 120 seconds before the operation of the foot SW.

The control section 63 outputs information about the amount of time shift to an event timing control section 75. A TC counter 76 gives, to the image processing section 65 and the event timing control section 75, time information as a time reference for encoding processing by the encoder 66. A time code of a medical image (movie) from the movie generation section 68 is specified based on the time information from the TC counter 76.

In the present embodiment, the event timing control section 75 determines a time code of a medical image duplicated by the movie duplication section 72 based on the time information from the TC counter 76 and information about the amount of time shift from the control section 63. The time codes have a common time axis between a medical image from the movie generation section 68 and a medical image duplicated by the movie duplication section 72, and to which time point in a medical image from the movie generation section 68 a medical image duplicated by the movie duplication section 72 corresponds is made clear.

The event timing control section 75 outputs determined time information to a meta-generation section 77. The meta-generation section 77 converts the inputted time information into meta-information. The meta-generation section 77 is configured to add meta-information including the time information to a medical image that is given and recorded in the HDD 70 so that the meta-information is recorded. Note that the time code is added to a medical image from the movie generation section 68 at the time of encoding or at the time of movie generation, but the meta-information, including the time information, from the meta-generation section 77 may be added to a medical image from the movie generation section 68 under the control of the event timing control section 75 so as to be recorded.

In a case where the movie duplication section 72 is caused to duplicate a medical image based on a state signal from an external appliance connected to the network 21, the control section 63 performs control such that the duplicated medical image is given to the movie generation section 73. In such a case, the control section 63 performs various types of setting such that the movie generation section 73 generates a movie suitable for a second use. In the case where a use for endoscopic surgical skill qualification is assumed as the second use, the movie generation section 73 generates a movie of a medium image quality in a DVD video format, for example. Note that the image quality is decided at the time of reading from the movie duplication section 72, and thus, in the case of causing the movie duplication section 72 to duplicate a medical image based on a state signal from an external appliance connected to the network 21, the control section 63 controls reading according to the use. For example, in the case where the electric scalpel device 13 is used, a medical image of a medium image quality and in a DVD format is outputted from the movie generation section 73 to the media driver 69.

However, as described above, with recording control performed based on a state signal from an external appliance connected to the network 21, a required scene is not necessarily recorded. Such a scene has to be specified by a surgeon without fail. Accordingly, when an operation signal is received at the UIIF 62, the control section 63 sets a third use. For example, when an operation signal is received at the UIIF 62, the control section 63 gives a medical image duplicated by the movie duplication section 72 to the movie generation section 74, and also, performs various types of setting so as to cause the movie generation section 74 to generate a movie suitable for the third use.

In the case where use for academic conference presentation is assumed as the third use, for example, the movie generation section 74 generates a movie of a high image quality of Full HD, for example. That is, in a case of causing the movie duplication section 72 to duplicate a medical image, when an operation signal is received at the UIIF 62, the control section 63 controls reading such that a medical image of a high image quality can be obtained. For example, when a surgeon operates the scope SW, the movie duplication section 72 reads a medical image at a high bit rate, and the movie generation section 74 outputs a medical image of a high image quality to the media driver 69.

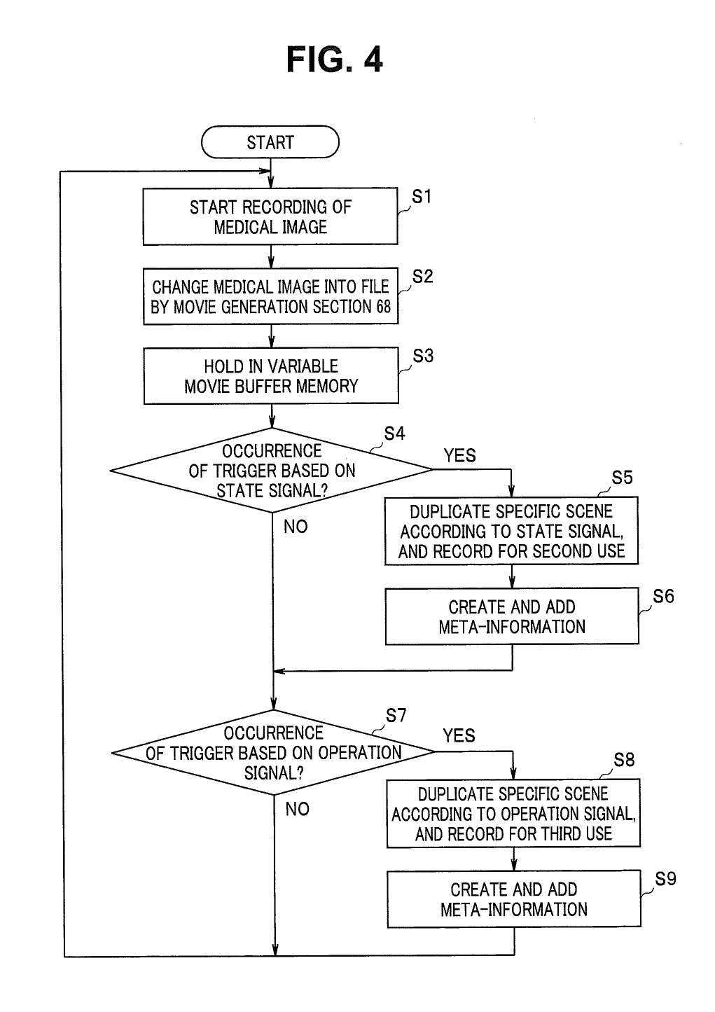

Next, an operation according to the embodiment having the configuration described above will be described with reference to the flowchart in FIG. 4.

The endoscope processor 15 generates an endoscopic image based on an image pickup signal from a not-shown endoscope. The endoscope processor 15 outputs the generated endoscopic image to the image recording device 60 as a medical image. In step S1, the image recording device 60 starts recording of the medical image. The medical image is captured by the image recording device 60 via the video IF 61, and is supplied to the encoder 66 of the image processing section 65. The encoder 66 outputs the inputted medical image after subjecting the medical image to predetermined encoding processing. For example, the encoder 66 performs an encoding process of generating an image at a maximum bit rate, such as an image of a Full HD image quality.

The output of the encoder 66 is supplied to the movie generation section 68. The movie generation section 68 generates a movie suitable for the first use from the inputted medical image. For example, the movie generation section 68 changes an image to be used as a record of evidence, which is the first use, such as a medical image of a low image quality, into a file (step S2), and outputs the result to the media driver 69. The media driver 69 records the inputted medical image in an external medium, and also, gives the medical image to the HDD 70 to have the medical image recorded. Moreover, the medical image from the encoder 66 is supplied, via the movie generation section 68, to the variable movie buffer memory 71 and is held in the variable movie buffer memory 71 (step S3).

In the next step S4, the control section 63 determines whether a trigger for movie recording has been generated or not, based on a state signal from an external appliance connected to the network 21. For example, the control section 63 determines that a trigger for movie recording has been generated, in a case where a change in a parameter of an external appliance is indicated by the state signal, such as in a case where start of endoscopic observation in a special light observation mode is indicated based a state signal from the light source device 16 or in a case where the electric scalpel device 13 is indicated based on a state signal from the electric scalpel device 13 to have reached a usable state, and then, the control section 63 proceeds to step S5 and instructs the movie duplication section 72 to duplicate the medical image.

The movie duplication section 72 reads from the variable movie buffer memory 71 a medical image at a time preceding by the amount of time shift set by the control section 63, and gives the medical image to the movie generation section 73. In such a case, the movie duplication section 72 performs reading in such a way that an image quality suitable for the second use, that is, around a medium image quality, is achieved, for example. The movie generation section 73 generates a movie in a format suitable for the second use from the inputted medical image, and gives the movie to the media driver 69. The media driver 69 gives the inputted medical image to the HDD 70 to have the medical image recorded.

On the other hand, the control section 63 outputs information about the amount of time shift to the event timing control section 75 at a timing of instruction of duplication, and the event timing control section 75 generates time information of the movie generated by the movie generation section 73 based on an output of the TC counter 76, and outputs the time information to the meta-generation section 77. The meta-generation section 77 converts the time information into meta-information, gives the meta-information to the HDD 70, and adds the meta-information to the movie generated by the movie generation section 73 (step S6). The media driver 69 reads the medical image, to which the meta-information is added, from the HDD 70, and records the medical image in an external medium.

Moreover, in step S7, the control section 63 determines, based on an operation signal received at the UIIF 62, whether a trigger for movie recording has been generated or not. For example, when a surgeon presses the scope SW at a scene he/she thinks important, the control section 63 determines that a trigger for movie recording has been generated, proceeds to step S8, and instructs the movie duplication section 72 to duplicate a medical image.

The movie duplication section 72 reads from the variable movie buffer memory 71 a medical image at a time preceding by the amount of time shift set by the control section 63, and gives the medical image to the movie generation section 74. In such a case, the movie duplication section 72 performs reading in such a way that an image quality suitable for the third use, that is, the Full HD image quality, is achieved, for example. The movie generation section 74 generates a movie in a format suitable for the third use from the inputted medical image, and gives the movie to the media driver 69. The media driver 69 gives the inputted medical image to the HDD 70 to have the medical image recorded.

Moreover, the event timing control section 75 generates time information of the movie generated by the movie generation section 74, based on information about the amount of time shift from the control section 63 and an output of the TC counter 76, and outputs the time information to the meta-generation section 77. The meta-generation section 77 converts the time information into meta-information, gives the meta-information to the HDD 70, and adds the meta-information to the movie generated by the movie generation section 74 (step S9). The media driver 69 reads the medical image to which the meta-information is added from the HDD 70, and records the medical image in an external medium. Note that the meta-generation section 77 may generate meta-information based on an ID of each use (use ID), and in steps S6, S9, meta-information indicating the use may also be added to the corresponding medical image.

As described above, in the present embodiment, a movie suitable for a predetermined use is generated from an output of the encoder, and also, at least one duplicate of a medical image is generated from the output of the encoder, and a movie suitable for a respective use is generated from the generated duplicate. Furthermore, start and end of recording of a movie based on a duplicate, and the use are controlled based on an operation signal from a surgeon or a state signal from an external appliance. A plurality of movies suitable for a plurality of uses may thereby be recorded at the same time. Moreover, a movie suitable for a predetermined use may be automatically recorded based on a state signal from an external appliance, and also, a movie suitable for a predetermined use may be recorded at an arbitrary timing based on an operation by a surgeon. A movie for a respective use may thus be recorded while preventing forgetting to record, and an editing task for obtaining a movie for a respective use after recording may be omitted. Furthermore, at the time of creating a duplicate of a medical image, a medical image may be recorded before a trigger for starting recording, and a required scene may be reliably recorded. Moreover, because meta-information according to time codes with a common time axis are added to medical images for respective uses, each medical image may be managed by a common time code. Accordingly, for example, reproduction of a medical image for a second use may be automatically started at the time of reproduction of a medical image for a first use.

Note that the first embodiment described above describes an example where a medical image for a first use is generated by the movie generation section 68, a medical image for a second use is generated by the movie generation section 73 based on a state signal from an external appliance, and a medical image for a third use is generated by the movie generation section 74 according to an operation signal based on an operation by a surgeon, but which of the movie generation sections 68, 73, 74 is to generate a medical image for which use is not particularly specified. Moreover, which of the movie generation sections 68, 73, 74 is to generate a movie according to which of a state signal from an external appliance and an operation signal based on an operation by a surgeon is not particularly specified.

### Second Embodiment

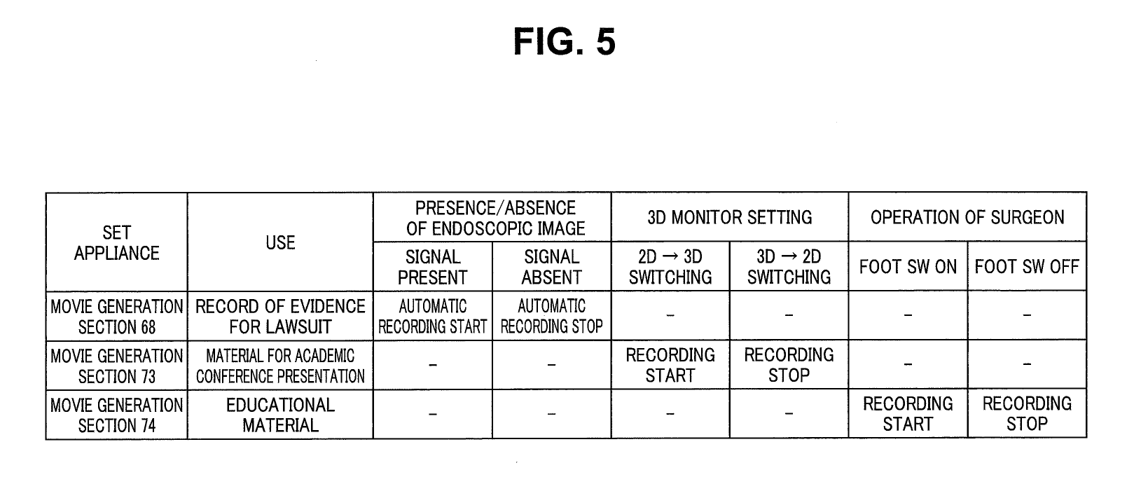

FIG. 5 is an explanatory diagram for describing a second embodiment of the present invention. A hardware configuration of the present embodiment is the same as the hardware configuration of the first embodiment. In the second embodiment, a playlist is prepared in advance, and setting may be freely performed with respect to uses to be assigned to a plurality of movie generation sections and determination of occurrence of a trigger.

FIG. 5 is an explanatory diagram for describing an example of a playlist used for setting of uses to be assigned to a plurality of movie generation sections and setting of a condition for determining occurrence of a trigger. The playlist can be stored in the memory 64, and according to the playlist read from the memory 64, the control section 63 controls assignment of a use, that is, controls generating a movie suitable for a use and decides a condition for determining occurrence of a trigger for a recording timing of a movie for the use.

The playlist in FIG. 5 shows an example where determination of presence/absence of an endoscopic image, setting of a 3D monitor, and an operation by a surgeon is performed as triggers for causing image recording. For example, when input of an endoscopic image via the video IF 61 is started, the control section 63 instructs the movie generation section 68 to generate a movie suitable for a use “record of evidence for lawsuit”, and causes recording of the movie to be automatically started. Furthermore, when the endoscopic image is no longer inputted via the video IF 61, the control section 63 causes recording of the movie to be stopped.

Furthermore, in a case of detecting, based on a state signal, that an endoscopic image is switched from being displayed on a not-shown 2D monitor to being displayed on a 3D monitor, the control section 63 instructs the movie generation section 73 to generate a movie suitable for a use “material for academic conference presentation”, and causes recording of the movie to be started. Moreover, in a case where an endoscopic image is switched from being displayed on the 3D monitor to being displayed on the 2D monitor, the control section 63 causes recording of the movie to be stopped.

Furthermore, in a case of detecting, by an operation signal, an operation by a surgeon to turn on the foot SW, the control section 63 instructs the movie generation section 74 to generate a movie suitable for a use “educational material”, and causes recording of the movie to be started. Moreover, in a case of detecting an operation by the surgeon to turn off the foot SW, the control section 63 causes recording of the movie to be stopped.

Note that the control section 63 is capable of updating the playlist when a not-shown input device is operated by a user.

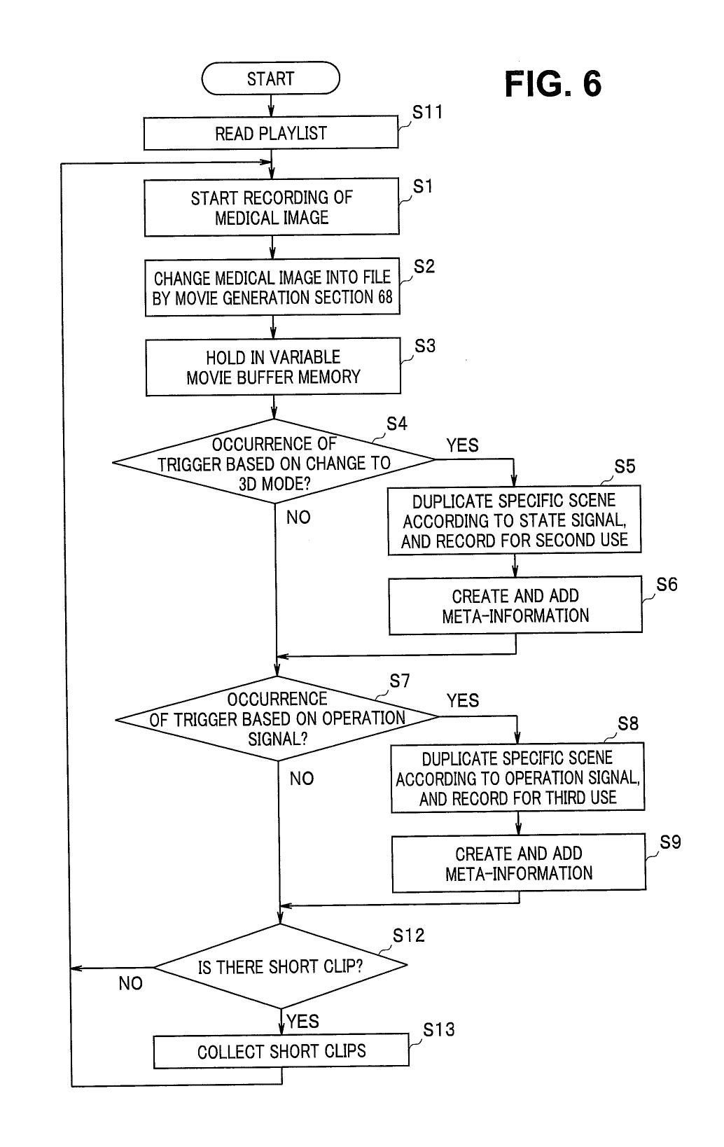

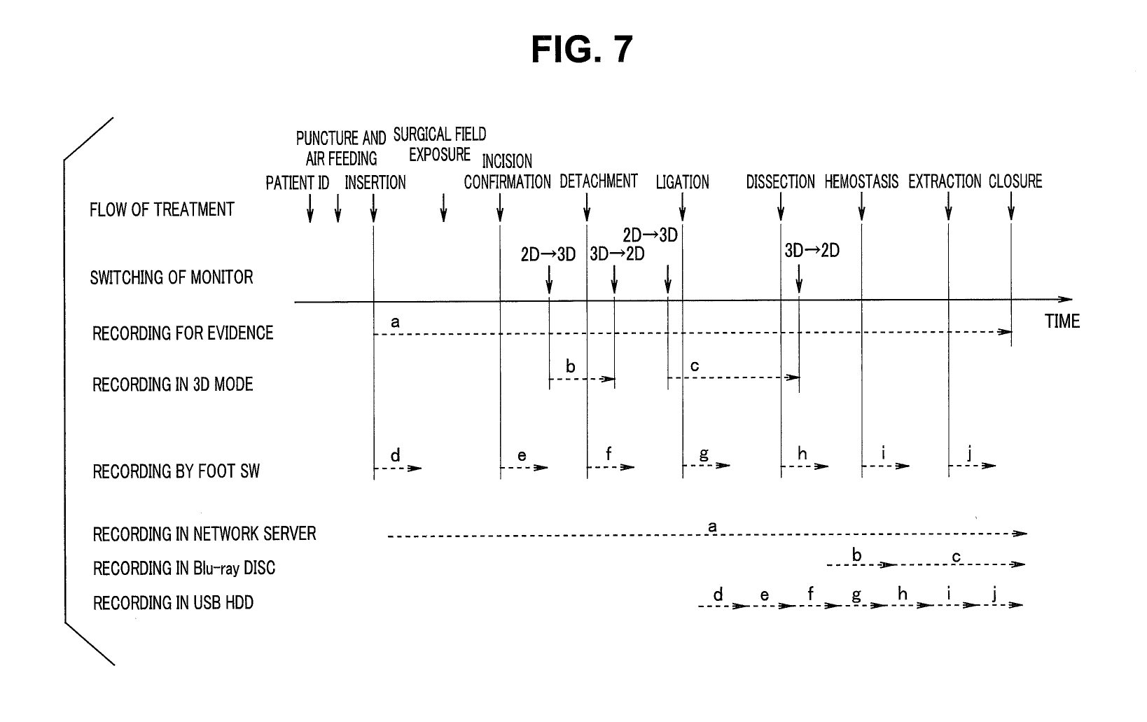

Next, an operation according to the embodiment having the configuration described above will be described with reference to FIGS. 6 and 7. FIG. 6 is a flowchart for describing an operation according to the second embodiment. In FIG. 6, the same steps as the steps in FIG. 4 are denoted by the same reference signs, and description of the steps is omitted. FIG. 7 is an explanatory diagram for describing the operation according to the second embodiment. FIG. 7 takes time as a horizontal axis, and shows a relationship between a flow of treatment in each procedure and a medical image to be recorded, while citing a laparoscopic surgery as an example.

In step S11 of the flow in FIG. 6, the control section 63 reads the playlist from the memory 64. According to the playlist which has been read, the control section 63 determines occurrence of triggers for start and end of recording of a medical image and performs recording control according to the use of a medical image to be recorded.

A description will be given assuming that a playlist of contents shown in FIG. 5 is stored in the memory 64, and that a laparoscopic surgery shown in FIG. 7 is performed. In FIG. 7, a start timing of each treatment and monitor mode switching are shown by arrows, and patient ID input, puncture and air feeding, endoscope insertion, surgical field exposure, incision confirmation, detachment, ligation, dissection, hemostasis, extraction, and closure are shown to be sequentially performed as treatments. Moreover, a monitor mode starts with a 2D mode, and a 3D mode is set only in a period of time including the detachment treatment and a period of time including litigation and dissection treatments.

In the example in FIG. 7, first, a task of inputting a patient ID is performed. Then, puncture and air feeding treatments are performed. Next, an endoscope is inserted. An image from the endoscope is supplied to the endoscope processor 15, and the endoscope processor 15 supplies the endoscopic image to the video IF 61 of the image recording device 60. The control section 63 instructs the movie generation section 68 to generate and record a medical image as a record of evidence for lawsuit in a manner shown in FIG. 5. Recording of a movie as evidence is thus started as shown by recording a indicated by a broken line in FIG. 7. The recording a is continued while the endoscopic image is being inputted, until a closure treatment is performed.

The control section 63 determines occurrence of a trigger based on a state signal in step S4 in FIG. 6, and determines occurrence of a trigger based on an operation signal in step S7. At the time of a surgeon inserting a scope, the foot SW is turned on for a predetermined period of time. Then, the control section 63 instructs the movie generation section 74 to generate a medical image as educational material in a manner shown in FIG. 5. Recording d of a movie for educational use is thus performed over a period of time indicated by a broken line in FIG. 7. In the same manner, the surgeon turns the foot SW on for a predetermined period of time as well as at the time of an incision confirmation treatment. Recording e of a movie for educational use is thus performed over a period of time indicated by a broken line in FIG. 7.

Next, in a detachment treatment, the endoscopic image is assumed to be switched from being displayed on the 2D monitor to being displayed on the 3D monitor. In such a case, based on a state signal from the monitor, the control section 63 instructs the movie generation section 73 to generate a medical image as material for academic conference presentation in a manner shown in FIG. 5. Recording of a movie for academic conference presentation is thus performed only during a period of time of display on the 3D monitor, as shown by recording b indicated by a broken line in FIG. 7.

Thereafter, in the same manner, recording of a movie for academic conference presentation is performed during a period of time when the endoscopic image is displayed on the 3D monitor, as shown by recording c indicated by a broken line in FIG. 7, and recording of a movie for educational use is performed during a period of time when the foot SW is turned on by the surgeon, as shown by recording f to j indicated by broken lines in FIG. 7. Note that the meta-generation section 77 adds time information to a medical image recorded in the HDD 70, based on an output of the event timing control section 75.

Furthermore, the meta-generation section 77 is capable of generating meta-information based on an ID of each use (use ID), and in steps S6, S9, meta-information indicating the use is also added to the corresponding medical image.

In the example in FIG. 6, in the case where a medical image recorded based on a state signal or a medical image recorded based on an operation signal (hereinafter such medical images will also be referred to as “short clip”) is present, the control section 63 proceeds from step S12 to step S13, and collects short clips assigned with the same use ID. For example, as shown in FIG. 7, the control section 63 causes the movie for a record of evidence (recording a) to be outputted to the network server, collectively records movies for academic conference presentation (recording b, c) in a Blu-ray disc, and collectively records movies for educational use (recording d to j) in an USB hard disk.

As described above, the present embodiment may also achieve the same effect as the first embodiment. Moreover, in the present embodiment, recording can be controlled according to a playlist set by a user as appropriate, and medical images corresponding to various uses may be generated at appropriate timings.

### (Modification)

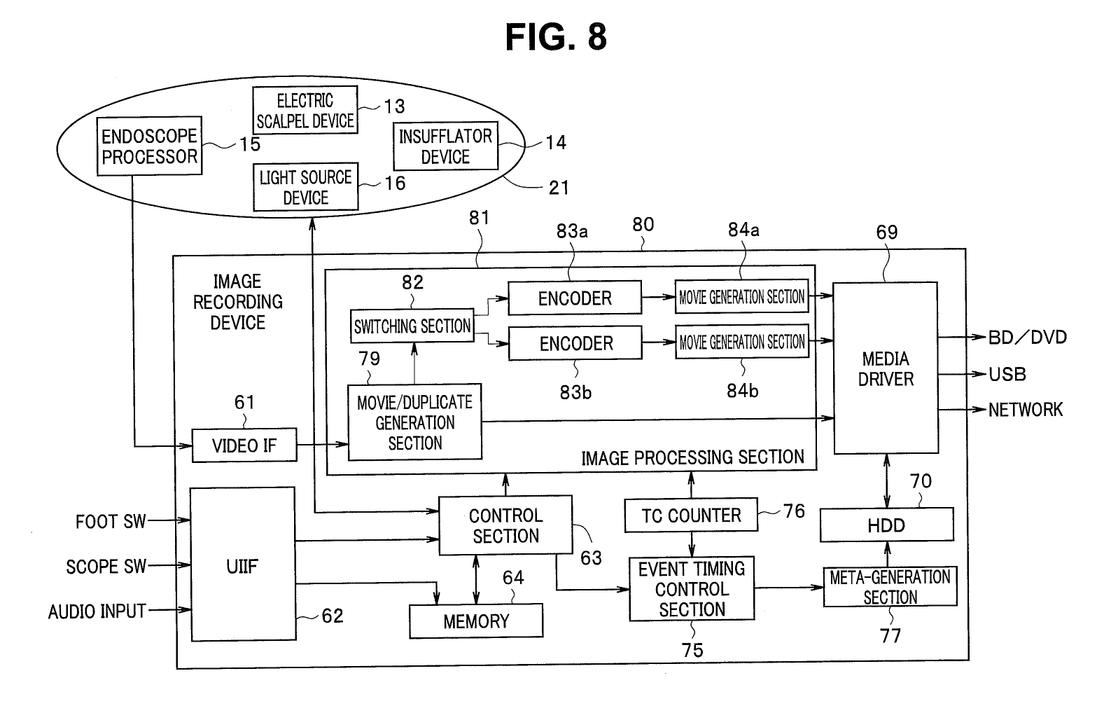

FIG. 8 is a block diagram showing a modification. In FIG. 8, the same structural components as the structural components in FIG. 1 are denoted by the same reference signs, and description of the structural components is omitted. In each of the embodiments described above, the movie generation sections 73, 74 are capable of outputting a movie of the same image quality as an output of the movie generation section 68, and are also capable of outputting a movie of an arbitrary image quality regardless of an output of the movie generation section 68 by performing re-encoding, for example. In the case where the movie generation sections 73, 74 include a re-encoding function, the encoding function may be realized by a separate circuit.

FIG. 8 shows an example of such a case, and an image processing section 81 of an image recording device 80 is configured of the movie/duplicate generation section 79, a switching section 82, encoders 83a, 83b, and movie generation sections 84a, 84b. An image (duplicate image) duplicated by the movie duplication section 72 of the movie/duplicate generation section 79 is given to the encoders 83a, 83b via the switching section 82. The switching section 82 is controlled by the control section 63 to supply the duplicate image to one or both of the encoders 83a, 83b.

The encoders 83a, 83b each store an inputted medical image in a not-shown frame memory, and also, read the medical image and perform encoding processing to thereby re-encode the inputted medical image into a video signal in a predetermined image format. For example, the encoders 83a, 83b are capable of re-converting an inputted medical image into a video signal of MPEG2 format, MPEG-4AVC/H.264 format, or the like. Medical images from the encoders 83a, 83b are given to the movie generation sections 84a, 84b, respectively.

Note that the movie generation section 68 may supply an image, of a highest image quality, outputted from the encoder 66 to the variable movie buffer memory 71, and reading of the movie duplication section 72 from the variable movie buffer memory 71 may be controlled such that a medical image with a maximum image quality can be outputted.

The movie generation section 84a is controlled by the control section 63 to generate a movie suitable for the second use based on the medical image encoded by the encoder 83a and to output the movie to the media driver 69, and the movie generation section 84b is controlled by the control section 63 to generate a movie suitable for the third use based on the medical image encoded by the encoder 83b and to output the movie to the media driver 69.

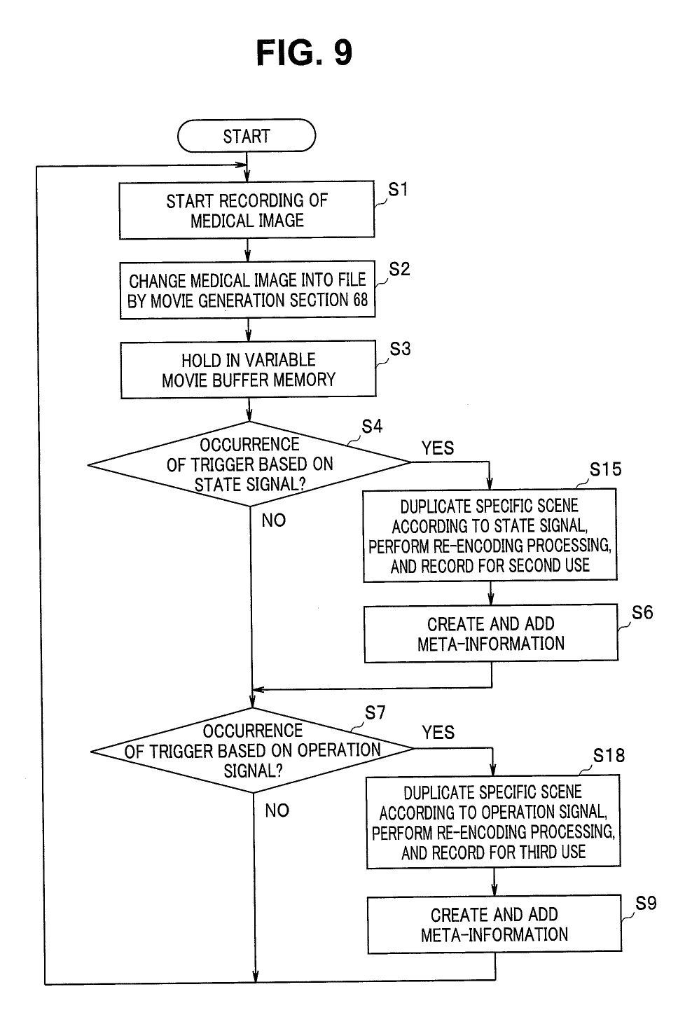

Next, an operation according to the modification having the configuration described above will be described with reference to the flowchart in FIG. 9. In FIG. 9, the same steps as the steps in FIG. 4 are denoted by the same reference signs, and description of the steps is omitted. The flow in FIG. 9 is different from the flow in FIG. 4 only in that steps S15, S18 for performing encoding processing are performed without fail instead of steps S5, S8. Note that FIG. 9 shows a flowchart corresponding to the first embodiment shown in FIG. 4, but the present modification may likewise be applied to the second embodiment in FIG. 6.

When determining, in step S4, that a trigger for recording a movie has been generated based on a state signal from an external appliance connected to the network 21, the control section 63 proceeds to step S15. In step S15, the movie duplication section 72 creates a duplicate of a medical image. The medical image from the movie duplication section 72 is supplied to the encoder 83a via the switching section 82. The encoder 83a subjects the inputted medical image to re-encoding processing. The encoder 83a is capable of generating a movie of a desired image quality under control of the control section 63. For example, the encoder 83a may generate a movie of around a medium image quality as the image quality suitable for the second use. The encoder 83a gives the encoded movie to the movie generation section 84a. The movie generation section 84a generates a movie in a format suitable for the second use based on the encoding result, and gives the movie to the media driver 69. The media driver 69 gives the inputted medical image to the HDD 70 to have the medical image recorded, for example.

Furthermore, in the case of determining, in step S7, that a trigger for starting recording has been generated based on an operation by a surgeon, the control section 63 proceeds to step S18. In step S18, the movie duplication section 72 creates a duplicate of the medical image. The medical image from the movie duplication section 72 is supplied to the encoder 83b via the switching section 82. The encoder 83b subjects the inputted medical image to re-encoding processing. The encoder 83b is capable of generating a movie of a desired image quality under control of the control section 63. For example, the encoder 83b may generate a movie of a Full HD image quality as the image quality suitable for the third use. The encoder 83b gives the encoded movie to the movie generation section 84b. The movie generation section 84b generates a movie in a format suitable for the third use based on the encoding result, and gives the movie to the media driver 69. The media driver 69 gives the inputted medical image to the HDD 70 to have the medical image recorded, for example.

Other effects are the same as the effects of each of the embodiments described above.

As described above, in the modification, re-encoding processing of a duplicated medical image using an independent encoder is possible. For example, a movie most suitable for an output medium may be obtained by conversion by the encoders 83a, 83b into an arbitrary image quality, resolution or the like, regardless of the image quality, the resolution or the like of the output of the movie generation section 68 of the movie/duplicate generation section 79. For example, in the case where the output medium is a tablet PC, recording suitable for a specified image size, capacity or the like that can be handled by the tablet PC is enabled by the encoding processing by the encoders 83a, 83b. In such a case, during a surgery, a surgeon may cause a movie of a high image quality for academic conference presentation to be recorded in a tablet PC to be used for academic conference presentation while causing a movie of a low image quality as a record of evidence to be outputted to the network server, for example.

### Third Embodiment

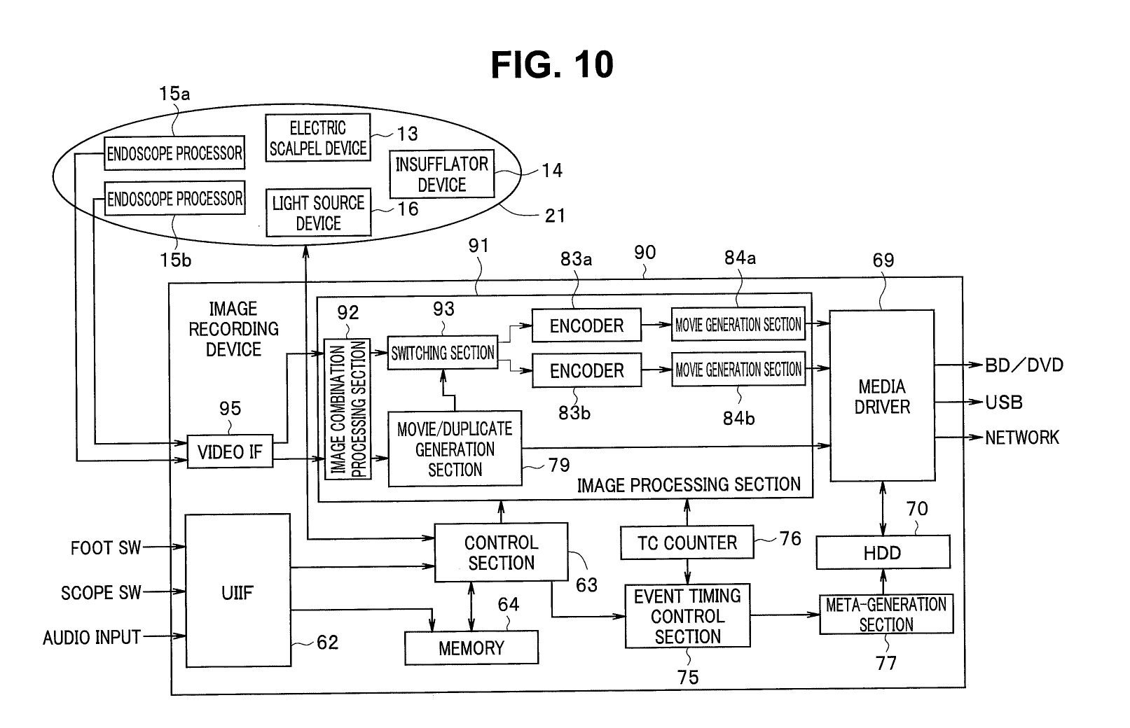

FIG. 10 is a block diagram for describing a third embodiment of the present invention. In FIG. 10, the same structural components as the structural components in FIG. 8 are denoted by the same reference signs, and description of the structural components is omitted. In each of the embodiments described above, the movie/duplicate generation section 79 encodes an inputted medical image of one system to generate a medical image for recording for the first use and to create a duplicate of the medical image, and enables generation of medical images for recording for the second and third uses based on the duplicate image. On the other hand, the present embodiment shows an example where inputted medical images of two systems can be handled.

Two endoscope processors 15a, 15b are provided in the network 21. In a surgery and the like, a plurality of endoscopes are sometimes used. For example, at the time of performing surgery on a digestive organ, such as stomach or duodenum, a digestive endoscope and a surgical endoscope are possibly used at the same time. In such a case, the endoscope processors 15a, 15b may capture an image from respective not-shown endoscopes, perform image signal processing, and output a medical image.

A medical image from each of the endoscope processors 15a, 15b is supplied to a video IF 95 of an image recording device 90. The video IF 95 is an interface suitable for image transfer, and captures medical images from the endoscope processors 15a, 15b of two systems. Note that various terminals such as a DVI (digital visual interface) terminal, an SDI (serial digital interface) terminal, an RGB terminal, a Y/C terminal, and a VIDEO terminal may be adopted for the video IF 95. The video IF 95 is capable of capturing various medical images of two systems not only from the endoscope processors 15a, 15b but also from an ultrasound device, a surgical field camera, an X-ray observation device, an endoscope processor different from the endoscope processors 15a, 15b, and the like.

Two medical images captured by the video IF 95 are given to an image combination processing section 92. By being controlled by the control section 63, the image combination processing section 92 may output the two medical images which are inputted, without any change, and may also combine and output the two medical images which are inputted. For example, the image combination processing section 92 may combine the images in a form of picture-out-picture (POP) according to which the two medical images which have been inputted are displayed next to each other, or may combine the images in a form of picture-in-picture (PIP) according to which one of the two medical images which have been inputted is displayed as a child image of the other image. The image combination processing section 92 may give one of the two inputted medical images or a combined image to the movie/duplicate generation section 79, and may give the other of the two inputted medical images or the combined image to a switching section 93. Note that the control section 63 may control combination processing of the image combination processing section 92 based on combination setting information stored in the memory 64, and combination switching setting information stored in the memory 64 may be changed as appropriate by user operation.

The movie/duplicate generation section 79 encodes an inputted combined image or medical image by the encoder 66. The movie/duplicate generation section 79 outputs a duplicate image based on an encoding result to the switching section 93, and also generates a movie based on the encoding result and outputs the movie to the media driver 69.

One of the two inputted medical images is given, without any change, to the switching section 93 from the image combination processing section 92, or a combined image of the two inputted medical images is given to the switching section 93. Moreover, a duplicated image is given to the switching section 93 from the movie/duplicate generation section 79. The switching section 93 is controlled by the control section 63 to give an image from the image combination processing section 92 to one of the encoders 83a, 83b, and to give an image from the movie/duplicate generation section 79 to the other of the encoders 83a, 83b. Alternatively, the switching section 93 may give an image from the movie/duplicate generation section 79 to both of the encoders 83a, 83b. Note that the control section 63 may control switching of the switching section 93 based on switching setting information stored in the memory 64, and the switching setting information stored in the memory 64 may be changed as appropriate by user operation.

The configuration is otherwise the same as the configuration of each of the embodiments described above.

In the embodiment having the configuration described above, a duplicate image is generated by the movie/duplicate generation section 79 based on one of two inputted medical images or a combined image of the two inputted medical images. Moreover, the movie/duplicate generation section 79 generates a movie for the first use based on one of the two inputted medical images or the combined image of the two inputted medical images (hereinafter such images will be referred to “master image”, in contrast to a duplicate image). The movie based on a master image is supplied by the movie/duplicate generation section 79 to the media driver 69.

The duplicate image from the movie/duplicate generation section 79 is given to the switching section 93. One of the two inputted medical images or the combined image of the two inputted medical images, that is, the master image, is given to the switching section 93 from the image combination processing section 92. The switching section 93 is controlled by the control section 63 to give the master image from the image combination processing section 92 to one of the encoders 83a, 83b and to give the duplicate image from the movie/duplicate generation section 79 to the other of the encoders 83a, 83b, or to give the duplicate image from the movie/duplicate generation section 79 to both of the encoders 83a, 83b.

The encoder 83a subjects the inputted image to encoding processing or re-encoding processing to obtain a movie of a desired image quality and the like, and outputs the movie to the movie generation section 84a. Moreover, the encoder 83b subjects the inputted image to encoding processing or re-encoding processing to obtain a movie of a desired image quality and the like, and outputs the movie to the movie generation section 84b. The movie generation section 84a gives the encoding result to the media driver 69 as a movie for the second use, and the movie generation section 84b gives the encoding result to the media driver 69 as a movie for the third use.

Movies suitable for maximum three uses are thus inputted to the media driver 69. That is, due to switching control by the switching section 93, movie(s) based on one or two master images and a movie based on one duplicate image are inputted to the media driver 69, or a movie based on one master image and movies based on two duplicate images are inputted to the media driver 69.

For example, an endoscopic image from a digestive endoscope and an endoscopic image from a surgical endoscope are assumed to be the medical images to be inputted to the video IF 95. In such a case, for example, the media driver 69 may be supplied with a movie for the first use based on the endoscopic image (master image) from the digestive endoscope, a movie for the second use based on the endoscopic image (master image) from the surgical endoscope, and a movie for the third use based on a duplicate image of the endoscopic image from the digestive endoscope. Furthermore, for example, the media driver 69 may be supplied with a movie for the first use based on a combined image (master image) of the two endoscopic images from the digestive endoscope and the surgical endoscope, a movie for the second use based on a duplicate image of the combined image of the two endoscopic images, and a movie for the third use based on a duplicate image of the combined image of the two endoscopic images. Moreover, for example, the media driver 69 may be supplied with a movie for the first use based on a combined image (master image) of the two endoscopic images from the digestive endoscope and the surgical endoscope, a movie for the second use based on a duplicate image of the combined image of the two endoscopic images, and a movie for the third use based on the endoscopic image (master image) from the digestive endoscope.

Furthermore, for example, the medical images to be inputted to the video IF 95 may be a 3D image and a 2D image based on an output of one 3D endoscope. In such a case, the media driver 69 may be supplied with a movie for the first use, of a low image quality and based on the 2D image (master image), a movie for the second use, of a high image quality and based on the 3D image (master image), and a movie for the third use, of a high image quality and based on a duplicate image of the 2D image.

As described above, in the present embodiment, one of two inputted medical images or a combined image of the two inputted medical images is taken as the master image, and movies based on two types of master images and a movie based on a duplicate image of one type of master image may be recorded, or a movie based on one type of master image and two types of movies based on a duplicate image of one type of master image may be recorded. Maximum three types of movies may be selected from many types of movies and be recorded by controlling combination processing for two medical images and switching processing for switching of inputs to encoders for the second and third uses.

Note that in the third embodiment, the switching section 93 can also be controlled such that one of two inputted medical images from the image combination processing section 92 or a combined image of the two inputted medical images are each given to both of the encoders 83a, 83b. In such a case, a movie based on a duplicate image cannot be recorded, but three types of movies based on the master image can be recorded.

### Fourth Embodiment

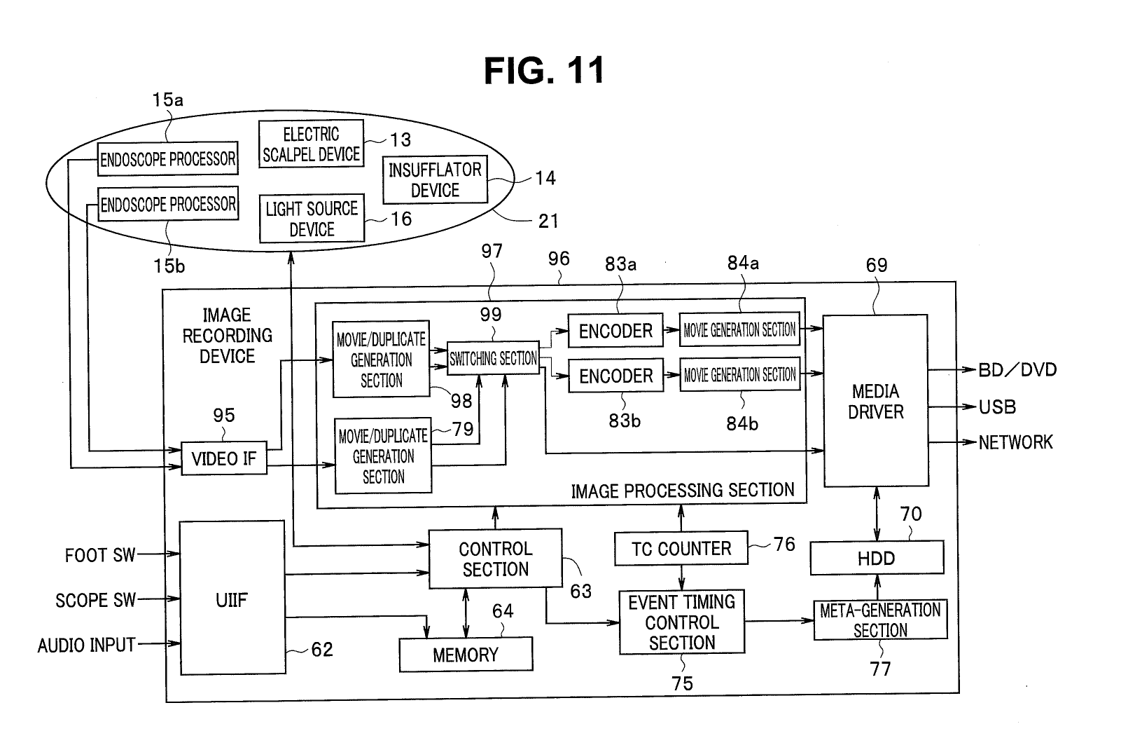

FIG. 11 is a block diagram for describing a fourth embodiment of the present invention. In FIG. 11, the same structural components as the structural components in FIG. 10 are denoted by the same reference signs, and description of the structural components is omitted. The present embodiment is different from the third embodiment in that the image combination processing section 92 is omitted, a movie/duplicate generation section 98 is added, and a switching section 99 is adopted instead of the switching section 93.

The video IF 95 gives one of two medical images, which are inputted, to the movie/duplicate generation section 79, and gives the other to the movie/duplicate generation section 98. The movie/duplicate generation section 98 is configured in the same manner as the movie/duplicate generation section 79. The movie/duplicate generation sections 79 and 98 both take an inputted medical image as the master image and generate a movie based on the master image, and also generate a duplicate image of the master image, and output the generated movie and duplicate image to the switching section 99.

The switching section 99 is controlled by the control section 63 to give three types of images, among four types of images, from the movie/duplicate generation sections 79, 98 to the encoders 83a, 83b and the media driver 69, respectively. Note that the switching section 99 is configured to select at least one duplicate image from four types of images, and to output the duplicate image.

Note that the control section 63 may control switching of the switching section 99 based on switching setting information stored in the memory 64, and the switching setting information stored in the memory 64 may be changed as appropriate by user operation.

The configuration is otherwise the same as the configuration of each of the embodiments described above.

In the embodiment having the configuration described above, the movie/duplicate generation section 79 takes one of two inputted medical images inputted to the video IF 95 as the master image, and generates a movie based on the master image and a duplicate image based on the master image. In the same manner, the movie/duplicate generation section 98 takes the other of the two inputted medical images as the master image, and generates a movie based on the master image and a duplicate image based on the master image. The four types of images generated by the movie/duplicate generation sections 79, 98 are supplied to the switching section 99. The switching section 99 is controlled by the control section 63 to give three inputted images including at least one duplicate image, among four inputs, to the encoders 83a, 83b or the media driver 69.

As described above, in the present case, either of two medical images may be taken as the master image, and a movie for any use may be generated by duplicating either of the two medical images as the master images.

Other effects and advantages are the same as the effects and advantages of each of the embodiments described above.

Note that the encoders can be omitted in the third and fourth embodiments.

Moreover, in each of the embodiments described above, an example is described where medical images suitable for three uses are generated and recorded, but medical images suitable for any number of uses may be simultaneously generated and recorded according to the number of movie generation sections.

The present invention is not limited to the respective embodiments described above, and structural components may be modified and embodied without departing from the gist in a stage of carrying out the invention. Moreover, various inventions can be made by combining a plurality of structural elements disclosed in the respective embodiments as appropriate. For example, some structural components among the entire structural components disclosed in the embodiments may be omitted. Moreover, structural components of different embodiments may be combined as appropriate.

What is claimed is:

1. An image recording device comprising:
a medical image capturing section configured to capture a medical image from a controlled appliance;
a single encoder configured to encode the medical image being inputted, into a video signal in a predetermined image format;
a first movie generation section configured to, based on the medical image encoded by the encoder and associated with reference time information, generate a first movie of a first image quality for a first use, and output a movie of a second image quality higher than the first quality, the movie of the second image quality being the inputted medical image;
a detection section configured to detect at least one of a first detection timing based on a state signal from an external appliance and a second detection timing based on an operation signal from an operation section;
a variable movie buffer memory configured to accumulate the movie of the second image quality outputted from the first movie generation section, over a predetermined period of time;
a duplication section configured to generate a duplicate of the movie of the second image quality accumulated in the variable movie buffer memory, at a detection timing detected by the detection section;
a second movie generation section configured to generate a second movie for a second use different from the first movie, based on the movie of the second image quality duplicated by the duplication section; and
a recording section configured to record the first movie, and to record the second movie in association with the reference time information.

2. The image recording device according to claim 1, wherein the movie of the second image quality is a movie of a highest image quality outputted from the encoder, and the first movie of the first image quality is a movie as a record for a lawsuit of a lower image quality than the second image quality.

3. The image recording device according to claim 1, further comprising a third movie generation section configured to generate a third movie for a third use based on the movie of the second image quality duplicated by the duplication section, wherein
the second movie generation section includes a processing section configured to generate the second movie for the second use based on the movie of the second image quality duplicated by the duplication section at the first detection timing detected by the detection section, and
the third movie generation section includes a processing section configured to generate the third movie for the third use based on the movie of the second image quality duplicated by the duplication section at the second detection timing detected by the detection section.

4. The image recording device according to claim 1, wherein the recording section includes
an event timing control section configured to determine a time in the movie generated by the second movie generation section, by being given the reference time information, and
a meta-generation section configured to generate meta-information based on the time determined by the event timing control section, and to record the meta-information in association with the movie generated by the second movie generation section.

5. The image recording device according to claim 4, wherein the event timing control section determines the time in the movie generated by the second movie generation section, based on the reference time information and information about the detection timing.

6. The image recording device according to claim 1, comprising:
a storage section configured to hold a playlist indicating a relationship between the state signal and movie generation control according to a use for recording of the medical image, and between the operation signal and the movie generation control; and
a control section configured to control the duplication section and the first and second movie generation sections according to the playlist.
